(19) United States
(12) Patent Application Publication
(10) Pub. No.: US 2008/0200632 A1
(43) Pub. Date: Aug. 21, 2008

(54) CATALYST, ITS PREPARATION AND USE

(76) Inventors: Ruth Mary Kowaleski, Cypress, TX (US); David Morris HAMILTON, Sugar Land, TX (US)

Correspondence Address: SHELL OIL COMPANY, P O BOX 2463, HOUSTON, TX, 772522463, US

(21) Appl. No.: 12/015,345

(22) Filed: Jan. 16, 2008

Related U.S. Application Data
(60) Provisional application No. 60/885,506, filed on Jan. 18, 2007.

(51) Int. Cl. B01J 23/745; B01J 23/72; B01J 23/83; B01J 23/58; B01J 23/64; C07C 5/333; C08F 12/02

(52) U.S. Cl. 526/346; 502/338; 502/331; 502/304; 502/330; 502/316; 502/328; 585/444

(57) ABSTRACT

A process for preparing a catalyst which process comprises preparing a mixture comprising iron oxide and at least one Column 1 metal or compound thereof, wherein the iron oxide is obtained by heating a mixture comprising an iron halide and at least 0.05 millimole of a metal chloride that is converted to a metal oxide under the heating conditions per mole of iron; a catalyst made by the above described process; a process for the dehydrogenation of an alkylaromatic compound which process comprises contacting the alkylaromatic compound with the catalyst; and a method of using an alkenylaromatic compound for making polymers or copolymers, in which the alkenylaromatic compound has been produced by the dehydrogenation process.

This application claims the benefit of U.S. Provisional Application No. 60/885,506, filed Jan. 18, 2007, which is hereby incorporated by reference.

### FIELD OF THE INVENTION

The present invention relates to a catalyst, a process for preparing the catalyst, a process for the dehydrogenation of an alkylaromatic compound and a method of using an alkenylaromatic compound for making polymers or copolymers.

### BACKGROUND

Iron oxide based catalysts and the preparation of such catalysts are known in the art. Iron oxide based catalysts are customarily used in the dehydrogenation of an alkylaromatic compound to yield, among other compounds, a corresponding alkenylaromatic compound. The dehydrogenation of alkylaromatic compounds is conventionally carried out on a commercial scale by passing an alkylaromatic feed and steam at an elevated temperature through a reaction zone containing a dehydrogenation catalyst. Steam is typically mixed with the alkylaromatic feed prior to its introduction into and contacting with the dehydrogenation catalyst of the reaction zone. The steam may serve as both a diluent and a heat source. As a heat source, the steam raises the temperature of the alkylaromatic feed to a dehydrogenation temperature, and it supplies the endothermic heat energy required by the resulting dehydrogenation reaction. As a diluent, the presence of steam in the reaction zone during the dehydrogenation reaction inhibits the formation and deposition on the dehydrogenation catalyst of carbonaceous residues. Typically, the stability and, thus, the useful life, of the dehydrogenation catalyst are improved with the use of a higher steam-to-oil ratio, which is defined as the ratio of the number of moles of steam to the number of moles of hydrocarbon, for example, ethylbenzene, fed to the reaction.

In this field of catalytic dehydrogenation of alkylaromatic compounds to alkenylaromatic compounds there are ongoing efforts to develop improved catalysts that may be made at lower costs. One way of reducing the cost of iron oxide based dehydrogenation catalysts is to use lower cost raw materials. Additional catalyst components are added to the iron oxide during catalyst preparation, and it is advantageous to use low cost raw materials as additional catalyst components. The additional catalyst components are typically metal oxides that serve various functions, for example as promoters and stabilizers. Metal chloride compounds are often less expensive than the corresponding metal oxide, and it would be advantageous to use metal chlorides as raw materials. One drawback of using metal chlorides is that residual chloride content in the catalyst has an adverse effect on catalyst performance. For example, residual chloride content can result in slower startup and a poorer initial catalyst activity.

Additionally, it is desirable from an energy savings standpoint to be able to operate a dehydrogenation process at as low of a steam-to-oil ratio as is possible. But, as suggested above, the operation of a dehydrogenation process at a reduced steam-to-oil ratio tends to cause the dehydrogenation catalyst to deactivate at an unacceptable rate thereby making the operation at such low steam-to-oil ratio commercially impractical. There have, however, been ongoing efforts to improve the operation and energy efficiency of dehydrogenation processes.

EP 1027928-B1 discloses catalysts containing iron oxide produced by the spray roasting of an iron salt solution. The iron oxide produced by the spray roasting process has a residual chloride content in the range of from 800 to 1500 ppm chloride. The iron oxide is typically combined with at least one potassium compound and one or more catalyst promoters to produce a catalyst. The patent discloses that a portion of the potassium compound and/or a portion of the promoters can for example be added to the iron salt solution used for spray roasting. This patent does not disclose a solution to the problem of residual chloride content or the adverse effect such residual chloride content may have on dehydrogenation catalyst performance.

### SUMMARY OF THE INVENTION

The invention provides a process for preparing a catalyst which process comprises preparing a mixture comprising iron oxide and at least one Column 1 metal or compound thereof, wherein the iron oxide is obtained by heating a mixture comprising an iron halide and at least 0.07 millimole of a non-iron metal chloride that is converted to a metal oxide under the heating conditions per mole of iron. The invention also provides a catalyst made by the above described process.

The invention further provides a process for the dehydrogenation of an alkylaromatic compound which process comprises contacting a feed comprising the alkylaromatic compound with a catalyst comprising iron oxide and at least one Column 1 metal or compound thereof wherein the iron oxide is obtained by heating a mixture comprising an iron halide and at least 0.07 millimole of a non-iron metal chloride that is converted to a metal oxide under the heating conditions per mole of iron.

The invention further provides a method of using an alkenylaromatic compound for making polymers or copolymers, comprising polymerizing the alkenylaromatic compound to form a polymer or copolymer comprising monomer units derived from the alkenylaromatic compound, wherein the alkenylaromatic compound has been prepared in a process for the dehydrogenation of an alkylaromatic compound using a catalyst comprising iron oxide and at least one Column 1 metal or compound thereof wherein the iron oxide is obtained by heating a mixture comprising an iron halide and at least 0.07 millimole of a non-iron metal chloride that is converted to a metal oxide under the heating conditions per mole of iron.

In a preferred embodiment, the invention provides a process for preparing a catalyst which process comprises preparing a mixture comprising doped regenerator iron oxide and at least one Column 1 metal or compound thereof wherein doped regenerator the iron oxide is obtained by adding copper or a compound thereof to an iron chloride mixture and heating the mixture.

In a further embodiment, the invention provides a process for preparing a catalyst which process comprises preparing a mixture comprising doped regenerator iron oxide and at least one Column 1 metal or compound thereof wherein the doped regenerator iron oxide is obtained by adding cerium or a compound thereof to an iron chloride mixture and heating the mixture.

### DETAILED DESCRIPTION OF THE INVENTION

The present invention provides a catalyst that satisfies the need for lower cost iron oxide based catalysts. The present invention also provides a catalyst that satisfies the need for iron oxide based dehydrogenation catalysts that operate effectively at low steam-to-oil conditions. The incorporation of additional catalyst components with the iron halide before it is heated eliminates the need to add those components after the iron oxide is formed. Additionally, some of the additional catalyst components may be added as chlorides without significantly increasing the residual chloride level in the iron oxide. Adding certain catalyst components before the iron oxide is formed may also result in improved catalyst performance, especially under low steam-to-oil conditions.

The iron oxide based dehydrogenation catalyst of the present invention is formed by mixing an iron oxide based catalyst precursor, hereinafter referred to as doped regenerator iron oxide, with additional catalyst components and calcining the mixture. The doped regenerator iron oxide is formed by heating a mixture comprising iron halide and a metal chloride to form the corresponding iron and metal oxides. As used herein, metal chloride refers to non-iron metal chlorides. In a preferred embodiment, the doped regenerator iron oxide is formed by spray roasting a mixture of iron halide and one or more metal chlorides to produce an iron oxide/metal oxide mixture.

The iron halide component of the iron halide/metal chloride mixture is preferably waste pickle liquor as generated by a steel pickling process. Waste pickle liquor is an acidic solution, typically comprising hydrochloric acid, which contains iron chloride. Alternatively, the iron halide may be present in dry or powder form or in an aqueous or acidic solution. The iron halide is preferably a chloride, but may also be a bromide. The iron may be at least partly present in a cationic form. The iron may be present in one or more of its forms including divalent or trivalent. An iron halide comprising chloride may be at least partly present as iron(II) chloride (FeCl₂) and/or iron(III) chloride (FeCl₃).

The metal chloride component of the iron halide/metal chloride mixture is any non-iron metal chloride that is converted to a metal oxide under the heating conditions necessary to convert at least a portion of the iron halide/metal chloride mixture to the corresponding oxides. A suitable metal chloride typically undergoes a hydrolysis reaction and an oxidation reaction to form the corresponding metal oxide. Suitable metal chlorides can be identified through experimentation, or they can be identified based on the value of the change in Gibbs energy of reaction (ΔGᵣₓₙ) for the reaction of the metal chloride with water and oxygen to form a metal oxide. The lower the ΔGᵣₓₙ, the more likely the conversion of the metal chloride to a metal oxide is to occur.

For example, under the heating conditions used to convert waste pickle liquor containing iron chlorides to iron oxide, those metal chlorides with values of ΔGᵣₓₙ that are lower or similar to the value of ΔGᵣₓₙ for converting FeCl₂ and/or FeCl₃ to Fe₂O₃ are especially suitable. If the ΔGᵣₓₙ of a metal chloride is significantly higher than the ΔGᵣₓₙ for converting FeCl₂ and/or FeCl₃ to Fe₂O₃, it is unlikely that the metal chloride will be converted to the corresponding metal oxide. This will result in a higher residual chloride content in the iron oxide, which leads to a slower catalyst startup and poorer initial catalyst activity. The conversion of the metal chloride to a metal oxide allows the additional catalyst components to be added as chlorides without resulting in a significantly increased residual chloride content of the regenerator iron oxide.

Examples of suitable metal chlorides include titanium, copper, cerium, manganese and zinc. The metal chloride may be at least partly present in a dry or powder form, or it may be at least partly present in solution. Further, the metal chloride may be at least partly present in a concentrated solution.

Additional catalyst components may also be added to the iron halide/metal chloride mixture to provide better incorporation of these components in the iron oxide/metal oxide mixture and it may reduce the complexity and cost associated with mixing and mulling the doped regenerator iron oxide with additional catalyst components during later catalyst preparation. Any additional catalyst component that does not impair the conversion of chlorides to oxides or otherwise negatively impact the heating of the iron halide/metal chloride mixture may be added at this stage.

For example, a lanthanide that is typically a lanthanide of atomic number in the range of from 57 to 66 (inclusive) may be added to the iron halide/metal chloride mixture. The lanthanide is preferably cerium. As additional examples, a Column 6 metal or compound thereof or titanium or a compound thereof may be added to the iron halide/metal chloride mixture. The additional catalyst component may be added to the iron halide/metal chloride mixture in a form that will convert to the corresponding oxide when heated.

Preparation of the Iron Halide/Metal Chloride Mixture may be carried out by any method known to those skilled in the art. The iron halide may be admixed or otherwise contacted with a metal chloride before the mixture is heated. In another embodiment, the iron halide may be admixed with a metal chloride during heating.

The iron halide/metal chloride mixture comprises at least 0.05 millimole of a metal chloride per mole of iron, preferably at least 0.07 millimole, more preferably at least 0.1 millimole, most preferably at least 5 millimole of a metal chloride per mole of iron. The mixture may comprise at most 200 millimole of a metal chloride per mole of iron, preferably at most 100 millimole, more preferably at most 50 millimole per mole of iron and most preferably at most 30 millimole per mole of iron.

In an embodiment wherein the mixture comprises titanium, the mixture may comprise from about 0.07 millimole to about 50 millimole of titanium per mole of iron. The mixture preferably comprises from about 3 to about 30 millimole of titanium, and more preferably comprises from 15 to about 20 millimoles of titanium per mole of iron

Once the iron halide/metal chloride mixture has been prepared, the mixture is heated to a temperature sufficient that at least a portion of the iron halide converts to iron oxide. The iron halide/metal chloride mixture may be present in gas, liquid, or solid form. The temperature may be sufficient such that at least part of any water and/or other liquids present evaporate. The temperature may be at least about 300° C., or preferably at least about 400° C. The temperature may be from about 300° C. to about 1000° C. or preferably from about 400° C. to about 750° C., but it may also be higher than about 1000° C. The heating may be carried out in an oxidizing atmosphere for example, air, oxygen, or oxygen-enriched air.

The mixture may be spray roasted as described in U.S. Pat. No. 5,911,967, which is herein incorporated by reference. Spray roasting comprises spraying a composition through nozzles into a directly heated chamber. The temperatures in the chamber may exceed 1000° C. especially in close proximity to the burner present in the directly heated chamber.

The above-described heating conditions for converting a metal chloride to a metal oxide may result in a portion of the metal chloride becoming volatile. This portion of volatile metal chloride would likely not be converted to metal oxide. The conditions may be adjusted to reduce volatilization of the metal chloride.

The doped regenerator iron oxide formed by the above-described heating may be present predominantly in the form of hematite (Fe₂O₃). The doped regenerator iron oxide may comprise iron oxide in any of its forms, including divalent or trivalent forms.

The doped regenerator iron oxide generally has a residual halide content of at most 3000 ppmw calculated as the weight of halogen relative to the weight of iron oxide calculated as Fe₂O₃, or at most 2000 ppmw, or at most 1500 ppmw, or at most 1250 ppmw, or preferably at most 1000 ppmw. The halide content may be at least 1 ppbw, at least 500 ppbw, or at least 1 ppmw. The halide is preferably chloride.

The doped regenerator iron oxide has a surface area that provides for an effective incorporation of catalyst components. The surface area of the doped regenerator iron oxide is generally at least 1 m²/g, preferably at least 2.5 m²/g, more preferably at least 3 m²/g, and most preferably at least 3.5 m²/g. As used herein, surface area is understood to refer to the surface area as determined by the BET (Brunauer, Emmett and Teller) method as described in Journal of the American Chemical Society 60 (1938) pp. 309-316.

The catalysts of the present invention may generally be prepared by any method known to those skilled in the art. Typically, the catalyst may be prepared by preparing a mixture comprising doped regenerator iron oxide, any other iron oxide(s), at least one Column 1 metal or compound thereof and any additional catalyst component(s), such as any compound referred to below, in a sufficient quantity. Further the mixture may be calcined. Sufficient quantities of catalyst components may be calculated from the composition of the desired catalyst to be prepared. Examples of applicable methods can be found in U.S. Pat. No. 5,668,075; U.S. Pat. No. 5,962,757; U.S. Pat. No. 5,689,023; U.S. Pat. No. 5,171,914; U.S. Pat. No. 5,190,906, U.S. Pat. No. 6,191,065, and EP 1027928, which are herein incorporated by reference.

Iron oxides or iron oxide-providing compounds may be combined with the doped regenerator iron oxide to prepare a catalyst. Examples of other iron oxides include yellow, red, and black iron oxide. Yellow iron oxide is a hydrated iron oxide, frequently depicted as α-FeOOH or Fe₂O₃.H₂O. At least 5 wt %, or preferably at least 10 wt % of the total iron oxide, calculated as Fe₂O₃, may be yellow iron oxide. At most 50 wt % of the total iron oxide may be yellow iron oxide. Additionally, black or red iron oxides may be added to the doped regenerator iron oxide. An example of a red iron oxide can be made by calcination of a yellow iron oxide made by the Penniman method, for example as disclosed in U.S. Pat. No. 1,368,748. Examples of iron oxide-providing compounds include goethite, hematite, magnetite, maghemite, lepidocricite, and mixtures thereof. Additionally, regenerator iron oxide that has not been prepared according to the invention may be combined with the doped regenerator iron oxide.

The quantity of the doped regenerator iron oxide in the catalyst may be at least 50 wt %, or preferably at least 70 wt %, up to 100 wt %, calculated as Fe₂O₃, relative to the total weight of iron oxide, as Fe₂O₃, present in the catalyst.

The Column 1 metal or compound thereof that is added to the catalyst mixture comprises a metal in Column 1 of the Periodic Table that includes lithium, sodium, potassium, rubidium, cesium and francium. One or more of these metals may be used. The Column 1 metal is preferably potassium. The Column 1 metals are generally applied in a total quantity of at least 0.2 mole, preferably at least 0.25 mole, more preferably at least 0.45 mole, and most preferably at least 0.55 mole, per mole iron oxide (Fe₂O₃), and typically in a quantity of at most 5 mole, or preferably at most 1 mole, per mole iron oxide. The Column 1 metal compound or compounds may include hydroxides; bicarbonates; carbonates; carboxylates, for example formates, acetates, oxalates and citrates; nitrates; and oxides.

Additional catalyst components that may be added to the doped regenerator iron oxide include one or more compounds of a Column 2 metal. Compounds of these metals tend to increase the selectivity to the desired alkenylaromatic compound, and to decrease the rate of decline of the catalyst activity. In preferred embodiments, the Column 2 metal may comprise magnesium or calcium. The Column 2 metals may be applied in a quantity of at least 0.01 mole, preferably at least 0.02 mole, and more preferably at least 0.03 mole, per mole of iron oxide calculated as Fe₂O₃, and typically in a quantity of at most 1 mole, and preferably at most 0.2 mole, per mole of iron oxide.

Further catalyst components that may be combined with the doped regenerator iron oxide include metals and compounds thereof selected from the Column 3, Column 4, Column 5, Column 6, Column 7, Column 8, Column 9, and Column 10 metals. These components may be added by any method known to those skilled in the art and may include hydroxides; bicarbonates; carbonates; carboxylates, for example formates, acetates, oxalates and citrates; nitrates; and oxides. Catalyst components may be suitable metal oxide precursors that will convert to the corresponding metal oxide during the catalyst manufacturing process.

The method of mixing the doped regenerator iron oxide and other catalyst components may be any method known to those skilled in the art. A paste may be formed comprising the doped regenerator iron oxide, at least one Column 1 metal or compound thereof and any additional catalyst component(s). A mixture may be mulled and/or kneaded or a homogenous or heterogeneous solution of a Column 1 metal or compound thereof may be impregnated on the doped regenerator iron oxide.

A mixture comprising doped regenerator iron oxide, at least one Column 1 metal or compound thereof and any additional catalyst component(s) may be shaped into pellets of any suitable form, for example, tablets, spheres, pills, saddles, trilobes, twisted trilobes, tetralobes, rings, stars, and hollow and solid cylinders. The addition of a suitable quantity of water, for example up to 30 wt %, typically from 2 to 20 wt %, calculated on the weight of the mixture, may facilitate the shaping into pellets. If water is added, it may be at least partly removed prior to calcination. Suitable shaping methods are pelletizing, extrusion, and pressing. Instead of pelletizing, extrusion or pressing, the mixture may be sprayed or spray-dried to form a catalyst. If desired, spray drying may be extended to include calcination.

An additional compound may be combined with the mixture that acts as an aid to the process of shaping and/or extruding the catalyst, for example a saturated or unsaturated fatty acid (such as palmitic acid, stearic acid, or oleic acid) or a salt thereof, a polysaccharide derived acid or a salt thereof, or graphite, starch, or cellulose. Any salt of a fatty acid or polysaccharide derived acid may be applied, for example an ammonium salt or a salt of any metal mentioned hereinbefore. The fatty acid may comprise in its molecular structure from 6 to 30 carbon atoms (inclusive), preferably from 10 to 25 carbon atoms (inclusive). When a fatty acid or polysaccharide derived acid is used, it may combine with a metal salt applied in preparing the catalyst, to form a salt of the fatty acid or polysaccharide derived acid. A suitable quantity of the additional compound is, for example, up to 1 wt %, in particular 0.001 to 0.5 wt %, relative to the weight of the mixture.

In one embodiment, the catalyst is formed as a twisted trilobe. Twisted trilobe catalysts are catalysts with a trilobe shape that are twisted such that when loaded into a catalyst bed, the catalyst pieces will not “lock” together. This shape provides a decreased pressure drop across the bed. Twisted trilobe catalysts are effective in dehydrogenation reactions whether they are formed with regenerator iron oxide, doped regenerator iron oxide, other forms of iron oxide or mixtures thereof. The mixture may be formed into a shape that results in a decreased pressure drop across a catalyst bed. Twisted trilobe catalysts are described in U.S. Pat. No. 4,673,664, which is herein incorporated by reference.

The catalyst mixture is preferably calcined. The calcination may comprise heating the mixture comprising doped regenerator iron oxide, typically in an inert, for example nitrogen or helium or an oxidizing atmosphere, for example an oxygen containing gas, air, oxygen enriched air or an oxygen/inert gas mixture. The calcination temperature is typically at least about 600° C., or preferably at least about 700° C. The calcination temperature will typically be at most about 1200° C., or preferably at most about 1100° C. Typically, the duration of calcination is from 5 minutes to 12 hours, more typically from 10 minutes to 6 hours.

The catalyst formed according to the invention may exhibit a wide range of physical properties. The surface structure of the catalyst, typically in terms of pore volume, median pore diameter and surface area, may be chosen within wide limits. The surface structure of the catalyst may be influenced by the selection of the temperature and time of calcination, and by the application of an extrusion aid.

Suitably, the pore volume of the catalyst is at least 0.01 ml/g, more suitably at least 0.05 ml/g. Suitably, the pore volume of the catalyst is at most 0.5, preferably at most 0.2 ml/g. Suitably, the median pore diameter of the catalyst is at least 500 Å, in particular at least 1000 Å. Suitably, the median pore diameter of the catalyst is at most 10000 Å, in particular at most 7000 Å. In a preferred embodiment, the median pore diameter is in the range of from 2000 to 6000 Å. As used herein, the pore volumes and median pore diameters are as measured by mercury intrusion according to ASTM D4282-92, to an absolute pressure of 6000 psia (4.2×10⁷ Pa) using a Micromeretics Autopore 9420 model; (1300 contact angle, mercury with a surface tension of 0.473 N/m). As used herein, median pore diameter is defined as the pore diameter at which 50% of the mercury intrusion volume is reached.

The surface area of the catalyst is suitably in the range of from 0.01 to 20 m²/g, more suitably from 0.1 to 10 m²/g.

The crush strength of the catalyst is suitably at least 10 N/mm, and more suitably it is in the range of from 20 to 100 N/mm, for example about 55 or 60 N/mm.

In another aspect, the present invention provides a process for the dehydrogenation of an alkylaromatic compound by contacting an alkylaromatic compound and steam with a doped regenerator iron oxide based catalyst made according to the invention to produce the corresponding alkenylaromatic compound. The dehydrogenation process is frequently a gas phase process, wherein a gaseous feed comprising the reactants is contacted with the solid catalyst. The catalyst may be present in the form of a fluidized bed of catalyst particles or in the form of a packed bed. The process may be carried out as a batch process or as a continuous process. Hydrogen may be a further product of the dehydrogenation process, and the dehydrogenation in question may be a non-oxidative dehydrogenation. Examples of applicable methods for carrying out the dehydrogenation process can be found in U.S. Pat. No. 5,689,023; U.S. Pat. No. 5,171,914; U.S. Pat. No. 5,190,906; U.S. Pat. No. 6,191,065, and EP 1027928, which are herein incorporated by reference.

The alkylaromatic compound is typically an alkyl substituted benzene, although other aromatic compounds may be applied as well, such as an alkyl substituted naphthalene, anthracene, or pyridine. The alkyl substituent may have any carbon number of two and more, for example, up to 6, inclusive. Suitable alkyl substituents are propyl (—CH₂—CH₂—CH₃), 2-propyl (i.e., 1-methylethyl, —CH(—CH₃)₂), butyl (—CH₂—CH₂—CH₂—CH₃), 2-methyl-propyl (—CH₂—CH(—CH₃) 2) and hexyl (—CH₂—CH₂—CH₂—CH₂—CH₂—CH₃), in particular ethyl (—CH₂—CH₃). Examples of suitable alkylaromatic compounds are butyl-benzene, hexylbenzene, (2-methylpropyl)benzene, (1-methylethyl)benzene (i.e., cumene), 1-ethyl-2-methyl-benzene, 1,4-diethylbenzene, in particular ethylbenzene.

The dehydrogenation process is typically carried out at a temperature in the range of from 500 to 700° C., more typically from 550 to 650° C., for example 600° C., or 630° C. In one embodiment, the dehydrogenation process is carried out isothermally. In other embodiments, the dehydrogenation process is carried out in an adiabatic manner, in which case the temperatures mentioned are reactor inlet temperatures, and as the dehydrogenation progresses the temperature may decrease typically by up to 150° C., more typically by from 10 to 120° C. The absolute pressure is typically in the range of from 10 to 300 kPa, more typically from 20 to 200 kPa, for example 50 kPa, or 120 kPa.

If desired, one, two, or more reactors, for example three or four, may be applied. The reactors may be operated in series or parallel. They may or may not be operated independently from each other, and each reactor may be operated under the same conditions or under different conditions.

When operating the dehydrogenation process as a gas phase process using a packed bed reactor, the LHSV may preferably be in the range of from 0.01 to 10 h⁻¹, more preferably in the range of from 0.1 to 2 h⁻¹. As used herein, the term “LHSV” means the Liquid Hourly Space Velocity, which is defined as the liquid volumetric flow rate of the hydrocarbon feed, measured at normal conditions (i.e., 0° C. and 1 bar absolute), divided by the volume of the catalyst bed, or by the total volume of the catalyst beds if there are two or more catalyst beds.

The conditions of the dehydrogenation process may be selected such that the conversion of the alkylaromatic compound is in the range or from 20 to 100 mole %, of from 30 to 80 mole %, or in the range of from 35 to 75 mole %, for example 40 mole %, or 67 mole %.

The alkenylaromatic compound may be recovered from the product of the dehydrogenation process by any known means. For example, the dehydrogenation process may include fractional distillation or reactive distillation. If desirable, the dehydrogenation process may include a hydrogenation step in which at least a portion of the product is subjected to hydrogenation by which at least a portion of any alkynylaromatic compound formed during dehydrogenation is converted into the alkenylaromatic compound. The portion of the product subjected to hydrogenation may be a portion of the product that is enriched in the alkynylaromatic compound. Such hydrogenation is known in the art. For example, the methods known from U.S. Pat. No. 5,504,268; U.S. Pat. No. 5,156,816; and U.S. Pat. No. 4,822,936, which are incorporated herein by reference, are readily applicable to the present invention.

Using a catalyst prepared according to the above-described process may decrease the selectivity of the dehydrogenation reaction to the alkynylaromatic compound. Accordingly, it may be possible to reduce the portion of the product that is subjected to hydrogenation. In some cases, the selectivity to the alkynylaromatic compound may be decreased to such an extent that the hydrogenation step may be eliminated.

The operation of a catalytic dehydrogenation process under low steam-to-oil process conditions can be desirable for a variety of reasons. But, the degree to which the steam-to-oil ratio may be reduced is typically limited by certain of the properties of the dehydrogenation catalyst used in the dehydrogenation process. In general, with the current economic considerations and commercially available dehydrogenation catalysts, the typical operation of a dehydrogenation process utilizes a steam-to-oil ratio exceeding 9:1, and, in most instances, the steam-to-oil ratio used is in the range exceeding 10:1. Many types of commercially available dehydrogenation catalysts even require the utilization of steam-to-oil ratios in the range exceeding 12:1 upwardly to 20:1.

As used herein, the steam-to-oil ratio is determined by dividing the number of moles of steam by the moles of hydrocarbon fed to the dehydrogenation reactor. The steam and hydrocarbon can be introduced separately to the reactor or can be mixed together first. A low steam-to-oil ratio is defined as a steam-to-oil ratio less than 9:1, preferably, less than 8:1, more preferably less than 6:1 and most preferably less than 5:1.

In one aspect, the invention comprises an improved method of manufacturing an alkenylaromatic, such as styrene, by the dehydrogenation of an alkylaromatic, such as ethylbenzene, involving the operation of a dehydrogenation process at a lower steam-to-oil process ratio than is typical. The utilization of a doped regenerator iron oxide based dehydrogenation catalyst formed according to the invention allows for the stable operation of a dehydrogenation process that is operated under low steam-to-oil process conditions. Also, such a dehydrogenation catalyst may provide for higher activity when used under low steam-to-oil process conditions.

There can be practical limitations on how low the steam-to-oil ratio may be reduced in the operation of the improved dehydrogenation process, since, much of the endothermic energy for the dehydrogenation reaction is supplied by the steam. Generally, the lower limit is no lower than 0.1:1 or 0.5:1 or even 1:1. Thus, for example, the improved dehydrogenation process may be operated at a steam-to-oil ratio in the range of from 0.1:1 to 9:1, preferably in the range of from 0.5:1 to 8:1, and most preferably from 1:1 to 6:1 or even from 1:1 to 5:1.

The alkenylaromatic compound produced by the dehydrogenation process may be used as a monomer in polymerization processes and copolymerization processes. For example, the styrene obtained may be used in the production of polystyrene and styrene/diene rubbers. The improved catalyst performance achieved by this invention with a lower cost catalyst leads to a more attractive process for the production of the alkenylaromatic compound and consequently to a more attractive process which comprises producing the alkenylaromatic compound and the subsequent use of the alkenylaromatic compound in the manufacture of polymers and copolymers which comprise monomer units of the alkenylaromatic compound. For applicable polymerization catalysts, polymerization processes, polymer processing methods and uses of the resulting polymers, reference is made to H. F. Marks, et al. (ed.), “Encyclopedia of Polymer Science and Engineering”, 2^{nd} Edition, new York, Volume 16, pp 1-246, and the references cited therein.

The following examples are presented to illustrate embodiments of the invention, but they should not be construed as limiting the scope of the invention.

### EXAMPLE 1

A copper-doped regenerator iron oxide (Doped) sample made by adding an aqueous solution containing approximately 2 moles of CuCl₂ per liter to a waste pickle liquor solution that contained approximately 3.7 moles of iron per liter was compared with a reference regenerator iron oxide (Ref) sample prepared without the addition of CuCl₂. Most of the iron was present as FeCl₂ and the waste pickle liquor solution contained approximately 150 g/L hydrochloric acid. The waste pickle liquor addition rate to the spray roaster was about 7.5 m³/h, and the copper chloride solution addition rate was adjusted to achieve the desired concentration of copper in the doped regenerator iron oxide. Due to the volatility of copper chloride, only a portion of the copper was retained in the iron oxide. The spray roaster was operated at typical spray roasting conditions known to those skilled in the art. The respective copper and chloride contents are shown in Table 1.

### EXAMPLE 2

A cerium-doped regenerator iron oxide (Doped) sample made by adding an aqueous solution containing approximately 2 moles of CeCl₃ per liter to a waste pickle liquor solution that contained approximately 3.7 moles of iron per liter was compared with a reference regenerator iron oxide (Ref) sample prepared without the addition of CeCl₃. The waste pickle liquor solution was added to a spray roaster as described in Example 1, and the cerium chloride solution addition rate was adjusted to achieve the desired concentration of cerium in the doped regenerator iron oxide. The respective cerium and chloride contents are shown in Table 1.

### EXAMPLE 3

A calcium-doped regenerator iron oxide (Doped) sample made by adding an aqueous solution containing approximately 3 moles of CaCl₂ per liter to a waste pickle liquor solution that contained approximately 3.7 moles of iron per liter was compared with a reference regenerator iron oxide (Ref) sample prepared without the addition of CaCl₂. The waste pickle liquor solution was added to a spray roaster as described in Example 1, and the calcium chloride solution addition rate was adjusted to achieve the desired concentration of calcium in the doped regenerator iron oxide. The respective calcium and chloride contents are shown in Table 1.

### EXAMPLE 4

A potassium-doped regenerator iron oxide (Doped) sample made by adding an aqueous solution containing approximately 0.6 moles of KCl per liter to a waste pickle liquor solution that contained approximately 3.7 moles of iron per liter was compared with a reference regenerator iron oxide (Ref) sample prepared without the addition of KCl. The waste pickle liquor solution was added to a spray roaster as described in Example 1 and the potassium chloride solution addition rate was adjusted to achieve the desired concentration of potassium in the doped regenerator iron oxide. The respective potassium and chloride contents are shown in Table 1.

TABLE 1

| Example | Ref Iron Oxide | Doped Iron Oxide | | Ref Iron Oxide | Doped Iron Oxide |
|---|---|---|---|---|---|
| No. | chloride (wt %) | chloride (wt %) | Metal | metal (wt %) | metal (wt %) |
| 1 | 0.057 | 0.095 | Cu | 0.006 | 0.160 |
| 2 | 0.093 | 0.117 | Ce | 0.005 | 1.270 |
| 3 | 0.121 | 1.252 | Ca | 0.004 | 0.814 |
| 4 | 0.088 | 0.326 | K | 0.002 | 0.260 |

The data in Table 1 shows that doping with metal chlorides (Such as CuCl₂ and CeCl₃) that are easily converted to oxides will not leave significant amounts of chloride in the iron oxide. On the other hand, use of dopants such as CaCl₂ and KCl that are not easily converted to oxides leads to retention of high levels of residual chloride in the iron oxide.

### EXAMPLE 5

Catalysts were prepared using the regenerator iron oxides of Example 1. Catalyst A was prepared using the following ingredients: 900 g of reference regenerator iron oxide of Example 1 and 100 g yellow iron oxide with sufficient potassium carbonate, cerium carbonate, molybdenum trioxide, and calcium carbonate to give a catalyst containing 0.516 mole K/mole Fe₂O₃, 0.022 mole Mo/mole Fe₂O₃, 0.027 mole Ca/mole Fe₂O₃, and 0.066 mole Ce/mole Fe₂O₃. Water (about 10 wt % relative to the weight of the dry mixture) was added to form a paste, and the paste was extruded to form 3 mm diameter cylinders cut into 6 mm lengths. The pellets were dried in air at 170° C. for 15 minutes and subsequently calcined in air at 825° C. for 1 hour. Catalyst B was prepared in the same manner as Catalyst A except that the copper-doped iron oxide of Example 1 was used in place of the reference regenerator iron oxide and the final catalyst contained 0.004 mole Cu/mole Fe₂O₃. Catalyst C was prepared in the same manner as Catalyst A using the reference regenerator iron oxide of Example 1, except that cupric chloride (CuCl₂.2H₂O) was added with the other catalyst ingredients to obtain a catalyst containing 0.004 mole Cu/mole Fe₂O₃.

A 100 cm³ sample of each catalyst was used for the preparation of styrene from ethylbenzene under isothermal testing conditions in a reactor designed for continuous operation. The conditions were as follows: absolute pressure 76 kPa, steam to oil (ethylbenzene) molar ratio of 10, and LHSV 0.65 h-1. In this test, the initial temperature was held at 600° C. The temperature was later adjusted such that a 70 mole % conversion of ethylbenzene was achieved (T70). The selectivity and conversion to styrene at the selected temperature were measured. The data is presented in Table 2.

TABLE 2

| | Catalyst A | | | Catalyst B | | | Catalyst C | | |
|---|---|---|---|---|---|---|---|---|---|
| Day | T ° C. | S % | Conv. % | T ° C. | S % | Conv % | T ° C. | S % | Conv. % |
| 3 | 600 | 95.9 | 66.6 | 600 | 96.3 | 65.3 | 601 | 97.2 | 55.7 |
| 8 | 600 | 95.0 | 71.0 | 599 | 94.5 | 73.6 | 601 | 96.6 | 57.0 |
| 13 | 597 | 95.2 | 70.8 | 591 | 95.1 | 70.1 | 596 | 95.0 | 66.4 |
| 22 | | | | | | | 595 | 95.3 | 70.2 |

The data in Table 2 show that Catalyst B prepared with copper-doped iron oxide starts up faster and results in better activity than Catalyst C made using the reference regenerator iron oxide in which the cupric chloride is added with the other catalyst ingredients during catalyst preparation.

Catalysts A, B, and C were also tested at a second set of conditions: a steam to oil (ethylbenzene) molar ratio of 5, absolute pressure of 40 kPa and LHSV 0.65 h⁻¹. The results for the catalysts after ten days of operation are shown in Table 3. This data shows that Catalyst B prepared with copper-doped iron oxide results in improved activity compared to Catalyst C made using the reference regenerator iron oxide in which the cupric chloride is added with the other catalyst ingredients during catalyst preparation or Catalyst A, which contains no added copper.

TABLE 3

| Catalyst A | | Catalyst B | | Catalyst C | |
|---|---|---|---|---|---|
| T70 ° C. | S70 % | T70 ° C. | S70 % | T70 ° C. | S70 % |
| 608.9 | 96.6 | 597.6 | 96.7 | 611.5 | 96.3 |

### EXAMPLE 6

Catalyst D was prepared using the reference iron oxide of Example 2 using the same procedures and ingredients described in Example 5. Catalyst E was prepared using the cerium-doped iron oxide of Example 2 by following the same catalyst preparation procedure, but less Ce₂(CO₃)₃ was added during catalyst preparation to compensate for the 0.013 mole Ce/mole Fe₂O₃ already present in the cerium-doped iron oxide. Catalyst F was prepared using the reference iron oxide and the same recipe as Catalyst D, except that a portion of the cerium (0.014 mole/mole Fe₂O₃) was added as CeCl₃ and the remainder (0.052 mole/mole Fe₂O₃) as Ce₂(CO₃)₃. All three catalysts contain a total cerium content of 0.066 mole Ce/mole Fe₂O₃.

The catalysts were tested at a steam to oil (ethylbenzene) molar ratio of 10 as described in Example 5, and results are shown in Table 4. The results show that Catalyst E, prepared with cerium-doped iron oxide, starts up faster and results in better selectivity and activity than Catalyst F made using the reference iron oxide in which the cerium was added as cerium chloride and cerium carbonate. In addition, Catalyst E shows improved selectivity at 70% conversion than Catalyst D made using the reference iron oxide in which the cerium was added solely as cerium carbonate.

TABLE 4

| | Catalyst D | | | Catalyst E | | | Catalyst F | | |
|---|---|---|---|---|---|---|---|---|---|
| Day | T ° C. | S % | Conv. % | T ° C. | S % | Conv. % | T ° C. | S % | Conv. % |
| 3 | 600 | 96.1 | 67.6 | 600 | 96.8 | 63.8 | 604 | 96.1 | 16.2 |
| 8 | 599 | 95.1 | 72.0 | 600 | 95.2 | 71.8 | 602 | 97.2 | 37.3 |
| 13 | 596 | 95.0 | 70.5 | 596 | 95.6 | 70.1 | 602 | 97.1 | 41.4 |
| 43 | | | | | | | 606 | 95.2 | 70.3 |

Catalysts D and E were also tested at a second set of conditions: a steam to oil (ethylbenzene) molar ratio of 5, absolute pressure of 40 kPa and LHSV 0.65 h⁻¹. The results for the catalysts after 10 days operation are shown in Table 5. This data shows that Catalyst E prepared with cerium-doped iron oxide results in improved activity and selectivity compared to catalyst D.

TABLE 5

| | |
|---|---|
| Catalyst D | Catalyst E |

| T70 ° C. | S70% | T70 ° C. | S70% |
|---|---|---|---|
| 611.5 | 96.1 | 605.4 | 96.7 |

### EXAMPLE 7

Catalyst G was prepared using the reference iron oxide of Example 3 by following the same procedure given for the reference iron oxide in Example 5. Catalyst H was prepared using the calcium-doped iron oxide using the same procedure, except no CaCO₃ was added during catalyst preparation so that the final Ca content in the catalyst was 0.029 mole/mole Fe₂O₃. Catalyst I was prepared like Catalyst G, except that CaCl₂.2H₂O was added during catalyst preparation instead of CaCO₃ so as to provide 0.033 moles Ca/mole Fe₂O₃ in the catalyst.

The catalysts were tested at a steam to oil (ethylbenzene) molar ratio of 10 as described in Example 5, and results are shown in Table 6. The results show that Catalyst H, prepared with calcium-doped iron oxide, starts up slowly and only achieves 37.5% conversion after 24 days of operation. Catalyst I, in which the calcium chloride has been added after iron oxide preparation with the other catalyst ingredients, shows similar slow startup behavior and low conversion. Catalyst G, in which calcium is added to the reference iron oxide as calcium carbonate along with the other ingredients, shows normal startup behavior and achieves 70% conversion within 8 days. The results show that the high level of chloride retained in the calcium-doped iron oxide used to prepare Catalyst H results in slow startup performance.

TABLE 6

| | Catalyst G | | | Catalyst H | | | Catalyst I | | |
|---|---|---|---|---|---|---|---|---|---|
| Day | T ° C. | S % | Conv. % | T ° C. | S % | Conv. % | T ° C. | S % | Conv. % |
| 3 | 602 | 96.4 | 62.2 | 604 | 96.8 | 15.8 | 604 | 96.9 | 18.4 |
| 8 | 601 | 94.9 | 71.6 | 603 | 97.4 | 25.9 | 600 | 97.3 | 26.0 |
| 13 | 595 | 94.9 | 70.2 | 602 | 97.5 | 28.5 | 599 | 97.3 | 32.0 |
| 24 | | | | 602 | 96.7 | 37.5 | | | |

### EXAMPLE 8

Catalyst J was prepared using the reference iron oxide of Example 4 by following the same procedure given in Example 5. Catalyst K was prepared using the potassium-doped iron oxide of Example 4 using the same procedure, except that the potassium carbonate added during catalyst preparation was reduced (to contribute 0.505 mole K/mole Fe₂O₃ in the catalyst) to supplement the potassium added as potassium chloride in the doped iron oxide. Catalyst L was prepared using the reference iron oxide of Example 4 using the procedure for Catalyst J, except that the potassium carbonate added during catalyst preparation was reduced (to give 0.505 mole K/mole Fe₂O₃ in the catalyst) and potassium chloride was added at the same level (0.011 mole K/mole Fe₂O₃) found in the doped iron oxide. All three catalysts J, K, and L contain the same level of total potassium (0.516 mole/mole Fe₂O₃)

The catalysts were tested at a steam to oil (ethylbenzene) molar ratio of 10 as described in Example 5, and the results are shown in Table 7. The results show that Catalyst K, prepared with potassium-doped iron oxide, starts up slowly and only achieves 54.2% conversion after 8 days of operation at around 600° C. Catalyst L, in which the potassium chloride has been added after iron oxide preparation with the other catalyst ingredients, shows similar slow startup behavior and low conversion. Catalyst J, in which potassium is added to the reference iron oxide as potassium carbonate along with the other ingredients, shows normal startup behavior and achieves 70% conversion within 8 days. The results show that the high level of chloride retained in the potassium-doped iron oxide used to prepare Catalyst K results in slow startup performance and poorer activity than Catalyst J.

TABLE 7

| | Catalyst J | | | Catalyst K | | | Catalyst L | | |
|---|---|---|---|---|---|---|---|---|---|
| Day | T ° C. | S % | Conv. % | T ° C. | S % | Conv. % | T ° C. | S % | Conv. % |
| 3 | 598 | 95.7 | 66.2 | 602 | 97.5 | 52.5 | 600 | 97.2 | 54.7 |
| 8 | 597 | 94.7 | 71.8 | 602 | 97.2 | 54.2 | 600 | 96.8 | 56.4 |
| 13 | 593 | 94.9 | 70.2 | 630 | 94.1 | 76.9 | 600 | 96.0 | 61.6 |
| 24 | | | | 603 | 95.7 | 70.3 | | | |

What is claimed is:

1. A process for preparing a catalyst which process comprises preparing a mixture comprising iron oxide and at least one Column 1 metal or compound thereof, wherein the iron oxide is obtained by heating a mixture comprising an iron halide and at least 0.07 millimole of a non-iron metal chloride that is converted to a metal oxide under the heating conditions per mole of iron.

2. A process as claimed in claim 1 wherein the mixture comprising an iron halide comprises from about 0.5 to about 100 millimoles of a non-iron metal chloride per mole of iron.

3. A process as claimed in claim 1 wherein the mixture comprising an iron halide comprises from about 2.5 to about 30 millimoles of a non-iron metal chloride per mole of iron

4. A process as claimed in claim 1 wherein the metal chloride comprises copper.

5. A process as claimed in claim 1 wherein the metal chloride comprises cerium.

6. A process as claimed in claim 1 wherein the Column 1 metal or compound thereof comprises potassium.

7. A process as claimed in claim 1 wherein the process further comprises adding a Column 2 metal or compound thereof to the mixture of iron oxide and Column 1 metal.

8. A process as claimed in claim 1 wherein the process further comprises adding yellow iron oxide to the mixture of iron oxide and Column 1 metal.

9. A process as claimed in claim 1 wherein the iron halide comprises an acidic solution of an iron chloride.

10. A process as claimed in claim 1 wherein the temperature of the heating is in the range of from about 300° C. to about 1000° C.

11. A process as claimed in claim 1 wherein the temperature of the heating is in the range of from about 400° C. to about 750° C.

12. A process as claimed in claim 1 wherein the heating comprises spray roasting.

13. A process as claimed in claim 1 wherein the process further comprises adding an additional catalyst component selected from the group consisting of cerium, molybdenum, tungsten, magnesium, calcium, copper, chromium, and compounds thereof to the mixture of iron oxide and Column 1 metal.

14. A process as claimed in claim 1 further comprising calcining the mixture at a temperature of from about 600° C. to about 1200° C.

15. A process as claimed in claim 1 further comprising calcining the mixture at a temperature of from about 700° C. to about 1100° C.

16. A catalyst prepared by the process of claim 1.

17. A process for the dehydrogenation of an alkylaromatic compound which process comprises contacting a feed comprising an alkylaromatic compound and steam with the catalyst of claim 16.

18. A process as claimed in claim 17 wherein the alkylaromatic compound comprises ethylbenzene.

19. A process as claimed in claim 17 wherein the steam-to-oil ratio of the process is less than 9:1.

20. A method of using an alkenylaromatic compound for making polymers or copolymers, comprising polymerizing the alkenylaromatic compound to form a polymer or copolymer comprising monomer units derived from the alkenylaromatic compound, wherein the alkenylaromatic compound has been prepared in a process for the dehydrogenation of an alkylaromatic compound as claimed in claim 17.

21. A process for preparing a catalyst which process comprises preparing a mixture comprising doped regenerator iron oxide and at least one Column 1 metal or compound thereof wherein the doped regenerator iron oxide is obtained by adding copper or a compound thereof to an iron chloride mixture and heating the mixture.

22. A process for preparing a catalyst which process comprises preparing a mixture comprising doped regenerator iron oxide and at least one Column 1 metal or compound thereof wherein the doped regenerator iron oxide is obtained by adding cerium or a compound thereof to an iron chloride mixture and heating the mixture.

23. A catalyst comprising doped regenerator iron oxide and potassium or a compound thereof wherein the doped regenerator iron oxide is obtained by heating an iron chloride compound in the presence of at least 5 millimoles of copper chloride per mole of iron.

24. A catalyst comprising doped regenerator iron oxide and potassium or a compound thereof wherein the doped regenerator iron oxide is obtained by heating an iron chloride compound in the presence of at least 5 millimoles of cerium chloride per mole of iron.
